(19) United States
(12) Patent Application Publication
(10) Pub. No.: US 2015/0112340 A1
(43) Pub. Date: Apr. 23, 2015

(54) FIXATION ROBOT FOR BONE FRACTURES

(71) Applicant: TAMIS OTOGAZ SANAYI TICARET VE MÜHENDISLIK HIZMETLERI ITHALAT INHRACAT LIMITED SIRKETI, Samsun (TR)

(72) Inventor: Hakan Kahvecioglu, Samsun (TR)

(73) Assignee: TAMIS OTOGAZ SANAYI TICARET VE MÜHENDISLIK HIZMETLERI ITHALAT INHRACAT LIMITED SIRKETI, Samsun (TR)

(21) Appl. No.: 14/381,369

(22) PCT Filed: Feb. 15, 2013
(86) PCT No.: PCT/TR2013/000062

§ 371 Date: Aug. 27, 2014

(30) Foreign Application Priority Data
Feb. 27, 2012 (TR) 2012/02175

(51) Int. Cl. A61B 17/64

(52) U.S. Cl. CPC A61B 17/6408
USPC 606/57

(57) ABSTRACT

A fixation robot enables the alignment of fractured bones in orthopaedic operations, wherein it includes the following: a fixed retainer jaw connected to one piece of the fractured bone, a moving retainer jaw connected to the other piece of the fractured bone and a motion transfer assembly providing motion to the moving retainer jaw.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The invention relates to a fixation robot, which performs the alignment of fractured bones automatically in the orthopedic operations carried out by means of an external fixation method and which is preferably controlled by means of a remote control.

2. Description of Related Art Including Information Disclosed Under 37 CFR 1.97 and 37 CFR 1.98

In the arm and leg fractures, when the physician decides to proceed with the external fixator treatment by taking the factors such as location and status of the fracture into account, the following known procedure is followed. Threaded nails are placed externally only on fractured bone fragments without making an incision over the fractured bones pieces Then, physician directs their assistants by looking at the screen of a so called fluoroscopy device (a camera showing live image by shooting X-ray video). Assistants acting on the order of the physician manually move the fractured bones pieces of the patient and try to align the fractured bones. However, in most operations, they cannot perform the reduction desired by the physician since they don't have enough power to overcome the resistance of the muscles around the fractured bone.

In the meantime, operating fluoroscopy device continuously and intensely emits X-ray (radiation). Although the patient receives this radiation once in their life, it is not a preferred situation for the physician to be exposed to this radiation. Thus, even though exact reduction desired is not attained, physician turns the fluoroscopy device off as soon as “enough” reduction is obtained. This situation increases the risk of never bonding or incorrect bonding of the fractured bones.

Even though the reduction is practised as the physician desired, assistants have to wait during one minute, which is the required time for the physician to secure the fixator (screwing the bolts), without changing their positions and the force they apply. In practice this is not quite possible as the staff become tired and reduces the force they apply unintentionally. Consequently, a poor or good reduction is provided, bolts of the fixator are tightened and the operation is terminated. External fixator is removed by the physician without the need for a second operation after bonding of the fractured bones at the end of the required time period and the treatment process is completed.

Patent no. WO 2002/069816 of Dutch origin is encountered during the patent search for the stabilization devices, so called fixator. The abstract of the patent application states the following: “The invention relates to an external fixation device for fractured bones having a carrying structure of interconnected elements comprising an extendible central body and clamps for bone screws respectively articulated on opposed ends of the central body.”

The other patent application is patent no. WO 2001/091654 of Italian origin, belonging to ORTHOFIX S.R.L. In this application the following is stated: “The invention relates to a new type of axial unilateral external splint device for stabilizing bone fractures, comprising an extendible rod-like middle body (2) and oppositely located bone screw clamps (5, 6) which are articulated to respective ends (3, 4) of the rod-like middle body (2) by means of ball joints. Advantageously, a ball-and-socket joint (16) is mounted to each clamp (5, 6), within amain body (20) with which a bone screw clamping arrangement (25, 26, 21, 22, 23) is associated or co-operates.”

The United States patent U.S. Pat. No. 5,662,648A discloses a fixation device having multi-axial movement and comprising a second mechanism in association with thereof through an intermediate member. Fixator secured on the bone by means of stabilizing elements (screws) is characterized in that it can be oriented with a second mechanism, however, in different axes, wherein a structure provided with freedom of movement by means of multi-axial movements is described.

### BRIEF SUMMARY OF THE INVENTION

The object of the present invention is to obtain a device having different technical specifications and bringing a new approach in this field as compared to the fixator configurations used in the current state of the art.

An object of the present invention is to provide bringing bone ends into the same axis or aligning them automatically by means of a remote control without manual intervention and adjustment.

Another object of the present invention is to protect physicians and other operating room personnel from the radiation emitted from fluoroscopy device during the operation by means of the remote control feature thereof.

Further object of the present invention are to exactly align the two bone pieces in order to prevent possibility of not bonding of the fractured bone or minimize the delay in bonding; and to provide immobility of the bones during the time period required for securing the fixator.

Another object of the present invention is to realize a precise alignment thanks to the structure comprising a fixed jaw and a moving jaw, with the moving jaw having the ability to move with minimum parameters and to be just fixed with the desired parameter.

Another object of the present invention is to provide high mobility thanks to the six-axis movement ability of the moving jaw.

A further object of the present invention is to provide easy transportation of the fixation robot thanks to the portable structure as well as the advancing means thereof.

Another object of the present invention is to minimize the treatment period of fractured bones and hence to carry out large number of treatments in shorter time periods. At the same time, the objective of the present invention is to minimize the effort spent by the physician and technical committee.

The present invention relates to a fixation robot enabling the alignment of fractured bones in orthopaedic operations for the purpose of fulfilling the objectives described above, wherein it comprises the following: a fixed retainer jaw connected to one piece of said fractured bone, a moving retainer jaw connected to the other piece of said fractured bone and a motion transfer assembly providing motion to said moving retainer jaw.

### BRIEF DESCRIPTION OF THE DRAWINGS

FIG. 1 is a representational front view of the fixation robot according to the invention when fixator and bone are adapted.

FIG. 2 is an individual perspective view of the fixation robot according to the invention.

FIG. 3 is a perspective view of the fixation robot according to the invention from a different angle.

FIG. 4 is a close-up perspective view of the cardan shaft transferring motion to the moving jaw of the fixation robot according to the invention.

### PART NUMBERS

10—Bone

11—Connection nail

12—Fixator

20—Fixation robot

21—Carrying body

22—Lower plate

23—Y-plane advancer

24—X-plane advancer

25—Z-plane advancer

26—Moving column

27—Driving member

28—Encoder

29—Cardan shaft

30—Moving retainer jaw

31—Fixed retainer jaw

32—Intermediate member

33—Fixed jaw arm

34—Wheels

35—Nail slots

40—Controller

A—Motion transfer assembly

### DETAILED DESCRIPTION OF THE INVENTION

The present invention relates to a fixation robot (20) enabling the alignment of fractured bones (10) in orthopaedic operations, wherein it comprises the following: a fixed retainer jaw (31) connected to one piece of said fractured bone (10), a moving retainer jaw (30) connected to the other piece of said fractured bone (10) and a motion transfer assembly (A) providing motion to said moving retainer jaw (30).

Said motion transfer assembly (A) comprises a Y-plane advancer (23) parallel to the ground; an X-plane advancer (24) provided at an angle of 90° with respect to the Y-plane advancer (23) of the motion transfer assembly (A); a Z-plane advancer (25) moving vertically with respect to ground of the motion transfer assembly (A); a moving column (26) (A); a plurality of driving members (27) in association with said moving retainer jaw (30), wherein said driving members (27) are a motor and a reducer. Motion transfer assembly (A) also comprises encoders (28).

The fixation robot comprises a cardan shaft (29) associated with moving retainer jaw (30); nails (11) anchored on said bone (10); a fixator (12) located between these nails (11) and jaws (30, 31); an intermediate member (32) connected with said fixed retainer jaw (31); a fixed jaw arm (33); a carrying body (21) where said motion transfer assembly (A) is mounted; a lower plate (22) provided below said carrying body (21); wheels (34); and a hand controller (40) enabling the movement of said motion transfer assembly (A) in 6-axis.

Operating Method of the Fixation Robot (20)

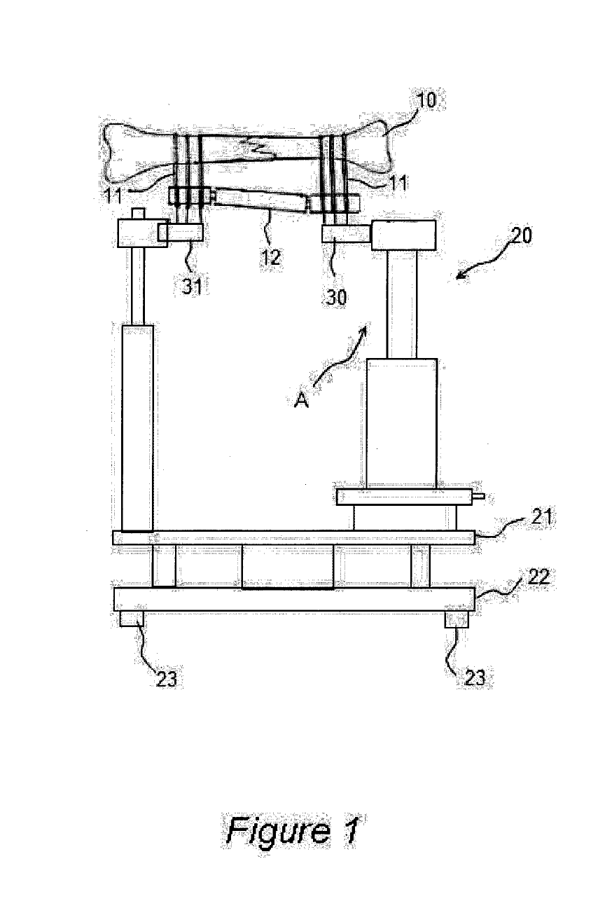

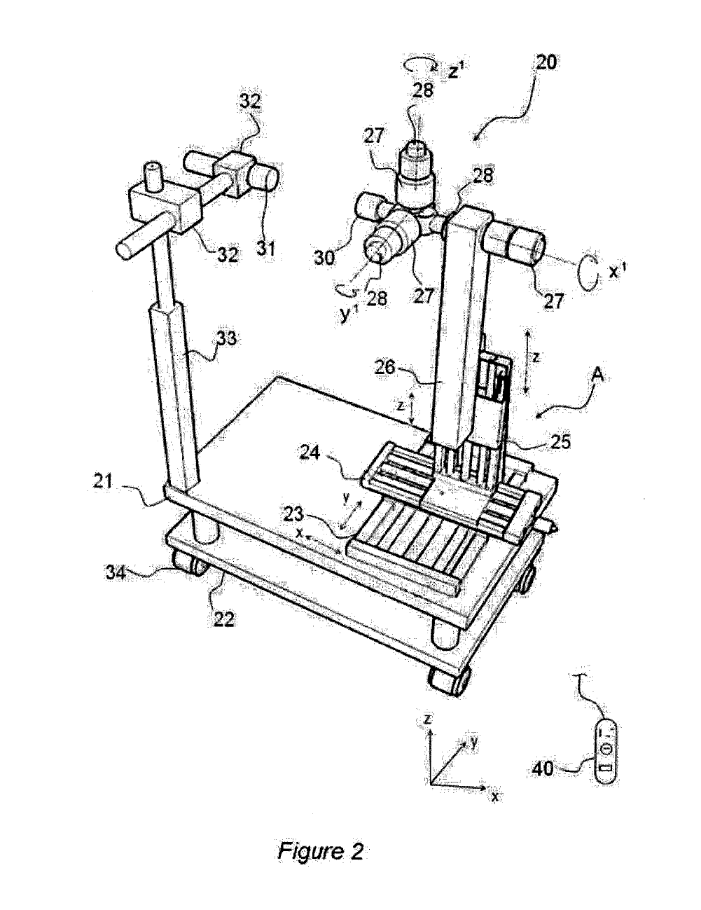

In FIG. 1, a representational two dimensional view of the fractured bone (10) held and fixed by the robot (20) is illustrated. One of the pieces of the two-piece fractured bone (10) is connected to the fixed retainer jaw (31) and the other one is connected to the moving retainer jaw (30) by means of nails (11) and/or different screw type connection members. Fixator (12) is preferably used between the bone (10) and the jaws (31, 30). The used fixator (12) is already an available fixator (12).

Moving retainer jaw (30) is provided on the carrying body (21) and it is capable of multi-axial movement. For example, the linear movement in y-plane is provided by the Y-plane advancer (23) while the movement in x-plane is provided by the X-plane advancer (24). The movement in the direction perpendicular to these planes (23, 24) is provided by the Z-plane advancer (25). Driving members (27) and cardan shaft (29) are provided on the Z-plane advancer (25) (see FIG. 4), enabling rotational movement of the moving jaw (30) in different axes.

Rotational movement of the moving retainer jaw (30) in the x′-axis is provided by the driving member (27) comprising a motor and reducer assembly. Axial rotational movement in the y′-axis and Z-axis is also provided by the same driving members (27). Driving members (27) are associated with the encoder (28), wherein the parameters are controlled by this encoder (28). Cardan shaft (29) provides the rotational movement in three axes (x′,y′,Z) by means of motors (see FIG. 4).

Fixed retainer jaw (31) is fixed on the carrying body (21) by means of intermediate members (32) and fixed jaw arm (33). Fixation robot (20) is provided with a portable construction and its transportation to any other location is possible by means of the wheels (34) thereof. This construction provides ease of use and practicality.

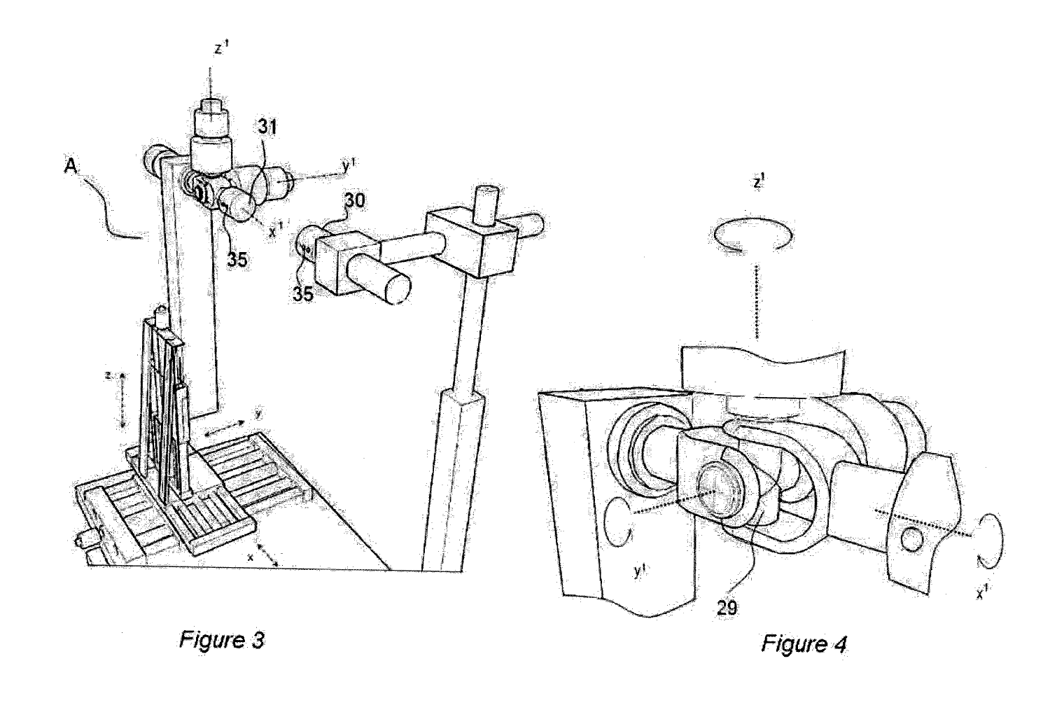

Nail slots (35) are formed on the moving and fixed retainer jaws (30, 31), wherein connection nails (11) providing the connection between the fixator (12) and bones (10) are located in said slots (35) (see FIG. 3). The fixator (12) used herein can be an intermediate component with a flexible structure as well as a mechanism having multiple joints.

1. A fixation robot enabling the alignment of fractured bones in orthopaedic operations comprising:
a fixed retainer jaw connected to one piece of said fractured bone;
a moving retainer jaw connected to the other piece of said fractured bone; and
a motion transfer assembly providing motion to said moving retainer jaw;
and characterized in that said motion transfer assembly comprises the following:
a Y-plane advancer parallel to the ground;
an X-plane advancer at an angle of 90° with respect to the Y-plane advancer and moving linearly on the Y-plane advancer;
a Z-plane advancer moving vertically with respect to the ground and moving linearly on the X-plane advancer;
a moving column carrying the moving retainer jaw and moving linearly on the Z-plane advancer; and
a cardan shaft associated with the moving retainer jaw and providing rotational movement to the moving retainer jaw around the x, y and z axes by being driven plurality of motors and reducers from different directions.

2. (canceled)

3. (canceled)

4. (canceled)

5. (canceled)

6. A robot according to claim 1, wherein it comprises a motion transfer assembly capable of moving in 6 axes comprising 3 degrees of translational movements and 3 degrees of rotational movements.

7. (canceled)

8. (canceled)

9. A robot according to claim 1, wherein said motion transfer assembly comprises encoders.

10. (canceled)

11. A robot according to claim 1, wherein it comprises nails anchored on said bone and a fixator provided between these nails and jaws.

12. A robot according to claim 1, wherein it comprises an intermediate member connected with said fixed retainer jaw and a fixed jaw arm.

13. A robot according to claim 1, wherein it comprises a carrying body onto which said motion transfer assembly is mounted.

14. A robot according to claim 12, wherein it comprises a lower plate and wheels located below the carrying body.

15. A robot according to claim 1, wherein it comprises a hand controller enabling the movement of motion transfer assembly in 6 axes comprising 3 degrees of translational movements and 3 degrees of rotational movements.

16. A robot according to claim 1, wherein it comprises nail slots formed on said jaws.

17. (canceled)

18. (canceled)
